(19) United States
(12) Patent Application Publication
(10) Pub. No.: US 2025/0388962 A1
(43) Pub. Date: Dec. 25, 2025

(54) RNA MAPPING METHODS WITH HIGH SEQUENCE COVERAGE

(71) Applicant: Waters Technologies Corporation, Milford, MA (US)

(72) Inventors: Martin Gilar, Franklin, MA (US); Catalin Doneanu, Franklin, MA (US)

(21) Appl. No.: 19/202,703

(22) Filed: May 8, 2025

Related U.S. Application Data
(60) Provisional application No. 63/644,263, filed on May 8, 2024.

(51) Int. Cl. C12Q 1/6872; C12Q 1/37; C12Q 1/6806; C12Q 1/6816

(52) U.S. Cl. CPC C12Q 1/6872; C12Q 1/37; C12Q 1/6806; C12Q 1/6816; G01N 2333/922

(57) ABSTRACT

Disclosed herein are methods of mapping a sequence of an RNA molecule comprising the steps of hybridizing a protecting primer with a portion the RNA molecule to form RNA/primer hybrid, combining the RNA molecule, the primer, and a digestion assay, digesting the RNA molecule hybridized with the primer in the digestion assay, and analyzing the two or more RNA fragments using liquid chromatography-mass spectrometry to determine the sequence of nucleotides in the RNA molecule. Also disclosed herein are digestion assays for selectively cleaving an RNA molecule into two or more RNA fragments.

### CROSS REFERENCE TO RELATED APPLICATIONS

This application claims the benefit of U.S. Provisional Application No. 63/644,263, filed May 8, 2024, which is hereby incorporated in its entirety by reference for all purposes.

### SEQUENCE LISTING

The instant application contains a Sequence Listing XML which has been submitted via Patent Center and is hereby incorporated herein by reference in its entirety. Said. XML copy, created on Jul. 30, 2025, is named “WAC-433US_SL.xml” and is 15,268 bytes in size.

### BACKGROUND

RNA sequences may be used for new therapeutic modalities for many healthcare applications, including vaccines and gene therapies. Mapping the exact sequence of the RNA nucleotides is required to ensure that the correct RNA sequences have been synthesized and can be used therapeutically. RNA sequences used in healthcare applications, such as mRNA, are typically between 2000 and 5000 nucleotides long. Current RNA mapping processes capable of detecting modified RNA sequences, however, are best suited for much shorter oligonucleotides that are between 6-20 nucleotides long, presenting significant challenges that can often lead to incomplete RNA mappings.

### SUMMARY OF THE DISCLOSURE

In one aspect, a method of mapping a sequence of an RNA molecule includes hybridizing a primer with a portion of the RNA molecule to form an RNA/primer hybrid having a protected RNA region hybridized with the primer and an unprotected RNA region, where the primer is selected from a group consisting of a synthetic DNA oligonucleotide, a morpholino DNA, and a PNA, digesting the RNA/primer hybrid in a digestion assay includes an enzyme configured to cleave a motif within the unprotected RNA region of the RNA/primer hybrid thereby forming two or more RNA fragments, and analyzing the two or more RNA fragments using liquid chromatography-mass spectrometry to determine a sequence of nucleotides in the RNA molecule. In some embodiments, the method also includes where the primer includes a sequence which is complementary with the protected RNA region and protects the protected RNA region from enzymatic cleavage. The method may also include where digesting is performed at a temperature between 15-24° C. The method may also include where the step of hybridizing the primer with the portion of the RNA molecule includes combining the primer with the RNA molecule in equimolar ratios so as to provide complete protection of the RNA molecule from enzymatic digestion. The method may also include where the step of hybridizing the primer with the portion of the RNA molecule includes combining the primer with the RNA molecule in sub-equimolar ratios so as to provide incomplete protection of the RNA molecule from enzymatic digestion. The method may also include where the primer has a length between 15-20 nucleotides. The method may also include where the primer is selected to provide an RNA/primer hybrid with a melting temperature between 60-70° C. The method may also include where the primer sequence extends from a first end to a second end, and where the primer hybridizes with a complementary portion of the RNA molecule from the first end to the second end, and where two or three nucleotides of the first end of the primer are configured to transition between being attached to and unattached from the complementary portion of the RNA molecule and where two or three nucleotides of the second end of the primer are configured to transition between being the attached and the unattached complementary portion of the RNA molecule. The method may also include includes hybridizing a second primer having a different sequence from the first primer with a second portion of the RNA molecule spaced apart from the portion of the RNA molecule hybridized with the first primer, where at least one of the first primer and the second primer is configured to protect a second motif in the RNA molecule which would produce a mononucleotide, a dinucleotide, and/or a trinucleotide interacting with the digestion assay. The method may also include where the primer is configured to hybridize with suspected mutation points within the RNA sequence The method may also include where the RNA molecule includes secondary and/or tertiary structures, and where the primer is configured to linearize the RNA molecule when hybridized so as to increase the susceptibility of the RNA molecule to enzymes during digestion compared to the RNA molecule unhybridized to the primer. The method may also include adding a buffer to the digestion assay, where the buffer is configured to stabilize the hybridizing of the primer with the RNA molecule relative to a digestion assay with no buffer. The method may also include further includes adding magnesium ions to the digestion assay at a concentration between 5-25 mM. The method may also include where the primer is functionalized with a highly retentive tag configured to modify the primer such that during liquid chromatography-mass spectrometry the primer elutes from a liquid chromatography column at a rate faster or slower than the rate of elution of the two or more RNA fragments. The method may also include where the primer is configured to protect 20-40% of the RNA molecule from enzymatic digestion. The method may also include where the digestion assay includes RNase T1. The method may also include where the digestion assay includes a ribonuclease enzyme selected from a group consisting of exoribonuclease I, exoribonuclease II, oligoribonuclease, polynucleotide phosphorylase (PNPase), RNase A, RNase Colicin E5, RNase cusativin, RNase D, RNase E, RNase H, RNase L, RNase MC1, RNase P, RNase PH, RNase PhyM, RNase R, RNase T, RNase T1, RNase T2, RNase U2, RNase V, and RNase III, or any combination of two or more thereof. The method may also include where the digestion assay includes one or more enzymes selected from a group consisting of Bal 31 endonuclease, colicin D, Endo R, eukaryotic nuclease enzymes, exoribonuclease I, exoribonuclease II, MazF, micrococcal nuclease, mung bean nuclease 1, Neospora endonuclease, oligoribonuclease, P1-nuclease, polynucleotide phosphorylase (PNPase), prokaryotic endonuclease enzymes, PrrC, RNase A, RNase Colicin E5, RNase cusativin, RNase D, RNase E, RNase enzymes, RNase H, RNase L, RNase MC1, RNase P, RNase PH, RNase PhyM, RNase R, RNase T, RNase T1, RNase T2, RNase U2, RNase V, RNase III, S1-nuclease, tRNAse-type nuclease enzymes, T4 endonuclease, T7 endonuclease, Ustilago nuclease, or any combination of two or more thereof. Other technical features may be readily apparent to one skilled in the art from the following figures, descriptions, and claims. In some embodiments, the method may also include where the buffer includes ammonium acetate (AmAc), triethylamine acetate (TEAAc), hexylamine acetate (HAAc), diisopropyl-ethylamine acetate (DIPEAAc), hexafluoropropanol (HFIP), sodium phosphate buffer (NaPhos), or any combination of two or more thereof. The method may also include where the buffer solution includes ammonium acetate (AmAc), HEPES (4-2-hydroxyethyl-1-piperazineethanesulfonic acid), hexafluoropropanol (HFIP), hexylamine acetate (HAAc), MOPS (3-(N-morpholino) propanesulfonic acid), PIPES (piperazine-N,N′-bis(2-ethanesulfonic acid)), sodium bicarbonate, Sodium phosphate (NaPhos), Triethylammonium acetate (TEAAc), Tris-acetate, Tris-base, Tris-Cl, and DBAA, diisopropyl-ethylamine acetate (DIPEAAc), or any combination of two or more thereof. The method may also include where the buffer has a concentration of at least 75 mM. The method may also include where the primer is functionalized with the highly retentive tag within three nucleotides of a first end, within three nucleotides of a second end, or functionalized within three nucleotides of the first end and the second end, respectively. Other technical features may be readily apparent to one skilled in the art from the following figures, descriptions, and claims.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “a protein” includes a single protein or a plurality of proteins.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used herein, the term “about” means within +10% of the value it modifies. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.” Unless otherwise clear from the context, all numerical values provided herein are modified by the term “about”.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

As used herein, the term “RNA sequence motif” refers to a specific nucleotide or specific sequence of nucleotides within an RNA sequence. In a non-limiting example, the RNA sequence motif may be “G,” “GG,” or “CA”. The RNA sequence motif may appear only once in an RNA sequence, or it may appear more than once at two locations within the RNA sequence.

As used herein, the terms “RNA” and “RNA sequence” refer to any type of RNA sequence including but not limited to a mRNA sequence, and a sgRNA sequence, and a tRNA primary sequence.

As used herein the terms “primer” and “protecting primer” refer to any type of molecule used to hybridize and/or couple with an RNA molecule to protect the coupled region from digestion such as enzymatic chevage. In non-limiting examples, a primer may comprise but is not limited to a synthetic DNA oligonucleotide, a DNA oligonucleotide, a morpholino DNA molecule, or a peptide nucleic acid (PNA).

As used herein the term “highly retentive tag” refers to chemical groups or modifications that can be attached to primers to enhance their retention in a liquid chromatography (LC) or high-performance liquid chromatography (HPLC) column.

As used herein, the terms “hybrid,” “hybridize,” “hybridized,” and “hybridizes,” refers to the process in which two complementary single-stranded DNA and/or RNA molecules bond together to form a double-stranded molecule (i.e., a hybrid). The bonding is dependent on the appropriate base-pairing across the two single-stranded molecules. As a non-limiting example, a portion of base pairs of an RNA molecule may hybridize with the base pairs in a DNA oligonucleotide primer to form an RNA/primer hybrid.

### BRIEF DESCRIPTION OF THE DRAWINGS

FIG. 1 illustrates an example of a liquid chromatography elution chromatogram of an exemplary RNA sequence after four minutes of digestion and after ninety minutes of digestion. The disappearance of many of the late-eluting peaks between the 4 minute and 90 minute digestion samples is indicative that the original RNA sequence is fully cleaved after 90 minutes and the partially cleaved sequences are further converted to fully cleaved digestion products, the short oligonucleotides.

FIG. 2 illustrates an example of a liquid chromatography elution chromatogram of an exemplary RNA sequence after four minutes of digestion with an RNase T1 endonuclease enzyme. The late-eluting peaks indicate partial digestion of the RNA sequence by the enzyme and offer an example of complete coverage of the RNA sequence.

FIG. 3 illustrates schematic view of a 50 nucleotide exemplary RNA molecule hybridized with an exemplary primer. FIG. 3 discloses SEQ ID NOS 12 and 16, respectively, in order of appearance.

FIG. 4 illustrates an example of a nano-differential scanning calorimetry thermogram used to measure the melting point of a 21/23 nucleotide RNA duplex in a 10 mM Na⁺ buffer solution. The melting point (Tₘ) is measured at the maximum point of the thermogram and the wide base of the peak thermogram peak is indicative of partial duplex melting below the Tₘ.

FIG. 5 illustrates an example of a bar graph showing the melting point of RNA duplexes held buffering solutions with different compositions and concentrations. Higher concentrations of buffer solutions and the addition of small cations like magnesium (Mg²⁺) increased the melting point and indicated increased stability of the RNA duplexes.

FIG. 6 illustrates an example of a liquid chromatography elution chromatogram of an exemplary 50 nucleotide long RNA molecule digested with and without a primer comprising DNA complementary oligonucleotides. The DNA complementary oligonucleotides hybridize with the RNA molecule and block an endonuclease enzyme from cleaving the duplex portion of the RNA molecule when hybridized with the primer.

FIG. 7 illustrates an example of a liquid chromatography elution chromatogram of an exemplary 80 nucleotide long RNA sequence digested with and without a primer comprising DNA complementary oligonucleotides. The DNA complementary oligonucleotides bind with the RNA and block the endonuclease enzyme from cleaving the RNA at the binding locations.

FIG. 8 illustrates a schematic view of the digestion products of the 50 nucleotide exemplary RNA molecule (SEQ ID NO: 13) hybridized with the primer of FIG. 6.

FIG. 9 illustrates a schematic view of an exemplary 80 nucleotide RNA molecule (SEQ ID NO: 14) hybridized with a first primer and a second primer of FIG. 7.

FIG. 10 illustrates an example of a high performance liquid chromatography elution of exemplary primers functionalized with retentive and highly retentive tags.

### DETAILED DESCRIPTION

Disclosed herein, in certain embodiments, are methods for mapping RNA sequences using liquid chromatography in combination with mass spectroscopy (LC-MS). RNA sequences may be designed for and delivered to a patient as therapeutic agents. For example, messenger RNA (mRNA) has emerged as a promising tool in the therapeutic landscape due to its ability to encode for specific proteins and elicit immune responses. For example, in vaccine development, mRNA may serve as a template in host cells of a patient for the expression of antigens, thereby enabling the immune system to recognize a pathogen and generate an immune response against the pathogen. Vaccine therapeutics including a targeted mRNA sequence are configured to deliver mRNA sequences which provide genetic instructions encoding target antigens into host cells, initiating the production of antigenic proteins to stimulate the immune system.

Furthermore, mRNA may be used in protein replacement therapy, where exogenous mRNA sequences are delivered to cells in order to compensate for deficient or malfunctioning proteins naturally occurring in a patient. This approach holds potential for treating a wide range of genetic disorders through utilizing host cells to produce proteins based on the exogenous mRNA sequences. mRNA-based protein replacement therapy offers a promising avenue for restoring cellular functions and ameliorating disease phenotypes. Therapeutic agents comprising RNA sequences such as mRNA offer several advantages over traditional therapeutic agents such as those used in vaccine therapeutics and treating protein malfunctions. For example, these therapeutics may improve development timelines, scalability, and versatility in antigen selection. In some embodiments, mRNA vaccines are engineered to incorporate modifications such as nucleoside modifications or lipid nanoparticle formulations to map and verify the RNA sequence, enhance stability, improve translational efficiency, and/or immunogenicity.

### Mapping RNA Sequences

In therapeutic treatments which comprise a primary sequence of RNA such as mRNA, sgRNA, and/or tRNA, it is important to confirm the primary sequence is a desired sequence prior to providing the therapeutic treatment to a patient. The desired sequence is the sequence of RNA intended to be included in the therapeutic treatment. An advantage of confirming the primary sequence is avoiding possibly health complications arising from delivering an RNA sequence which encode a different protein than the desired protein elucidating the therapeutic effect. For example, an RNA sequence encoding a protein other than the desired protein may have negative health effects on the patient and/or may not provide the intended therapeutic effect (e.g., an immune response or replacing a malformed or deficient protein).

mRNA sequences used as or within a therapeutic agent typical have a length between 2000-5000 nucleotides and a molecular weight between 0.6-1.5 MDa. Molecules within this size range may be challenging to characterize using traditional separation methods or mass spectrometry because of their long length. Other types of RNA sequences such as sgRNA which may be used for CRISPR/Cas9 therapy are typically about 100 nucleotides long.

Traditional NGS (next generation sequencing) methods may present challenge because they are expensive, require complex bioinformatic software, and do not distinguish between native and chemically modified nucleotides in the primary sequence. In some embodiments, the RNA sequence includes one or more modifications such as nucleobase methylation, ribose 2′O methylation, and/or RNA backbone phosphorothioate modification. However, NGS methods may be unable to distinguish these modifications from their native (unmodified) counterparts.

Separation and detection methods such as using LC-MS may be used to detect DNA and/or RNA oligonucleotides modifications in their sequence, because these modifications have a different mass than their native counterparts. In some embodiments, a second system is used to confirm the sequence of short RNA fragments. For example, the RNA fragments may be analyzed with a tandem mass spectrometry (MS/MS) to perform confirmatory sequencing of short RNA molecules.

In some embodiments, the method includes confirming the sequence of one or more RNA molecules and/or fragments by comparing the resulting profile from the LC-MS alone or in combination with MS/MS with the profile of one or more known RNA sequences. For example, the measured profile may include the mass of fragments, the retention times of fragments which may be compared with the mass of known RNA sequences, the retention times of known RNA sequences, the theoretical RNA sequence, and/or the theoretical retention times. In some embodiments, the sequence of the measured RNA is confirmed if the profile of the measured RNA shares a sequence between 60-70%, 70-80%, 80-90%, 90-95%, or 95-99% with the known and/or theoretical RNA.

In some embodiments, the method of confirming and/or determining a sequence of RNA includes digesting an RNA molecule having a length greater than 30 nucleotides into two or more RNA fragments. In some embodiments, the method includes adding the RNA molecule to a digestion assay comprising an enzyme capable of cleaving at least a portion of RNA molecule to form the two or more RNA fragments.

Since RNA consist of only four basic building units (A, C, U, G mononucleotides), digestion assays configured to digest RNA molecules into RNA fragments may result in two or more RNA fragments having the same mass. For example, the digestion assay may comprise isobaric oligonucleotide RNA fragments such as ACAA and AACA, making it difficult to distinguish between the two fragments and/or determine the RNA sequence. Moreover, digestion assays may result in multiple identical short sequences in the RNA sequence which makes the sequencing ambiguous, unless those motifs are part of longer RNA oligonucleotides of unique mass and sequence.

In some embodiments, the method includes measuring the profile of the two or more RNA fragments. In some embodiments, each RNA fragment forms an oligonucleotide having a length of about 20 nucleotides long. In some embodiments, each oligonucleotide is between 5-10, 10-15, 15-20, 20-25, and 25-30 nucleotides long. In some embodiments, each oligonucleotide is between 5-15, and 15-30 nucleotides long. In some embodiments, each oligonucleotide is between 5-30 nucleotides long. In some embodiments, each oligonucleotide is between 6-20 nucleotides long. In some embodiments, each oligonucleotide is between 6-30 nucleotides long. A person having skill in the art would appreciate that oligonucleotides longer than 30 nucleotides long may result in the sequence read as incomplete. Likewise, oligonucleotides shorter than 5 nucleotides long may be non-informative, as such sequences motifs can occur in many positions of the original RNA sequence and may produce similar MS reading as other short oligonucleotides and therefore difficult to identify.

### Digestion Assays

Traditional protein top-down sequencing of peptide mapping method utilizes endonucleases or other high-fidelity enzymes to map the sequence of an RNA molecule. However, these top-down methods, are limited to a few high-fidelity enzymes capable of cleaving single stranded RNA molecules with high selectivity and fidelity and are more likely to have RNA fragments having longer than 20 nucleotide residue lengths, which are difficult to sequence by MS/MS to arrive at a complete sequence. Likewise, less highly selective enzymes are more likely to result in fragments that are too short and have the same sequence and/or the same mass, thus making it difficult to accurately map the RNA sequence.

Embodiments described herein include digestion assays comprising enzymes capable of digesting RNA sequences, into two or more RNA fragments for RNA sequence mapping. Typically, enzymes used in digestion assays are selected to digest (e.g., cleave) a bond at a desired location between two adjacent ribonucleotides, where the desired location corresponds with an RNA sequence motif within the RNA. In some embodiments, any enzyme that digests (e.g., cleaves) bonds between ribonucleotides, for example, a nuclease enzyme or a ribonuclease enzyme, is used in the methods described herein. In some embodiments, a nuclease enzyme or a ribonuclease enzyme is used in the methods described herein. In some embodiments, a digestion assay is used to digest an RNA sequence into two or more RNA fragments.

In some embodiments, one or more enzymes are included in the digestion assay such that the resulting RNA sequence fragments are short oligonucleotides having a length between 1-3, 3-5, 5-7, 7-10, 10-13, 13-15, 15-17, 17-19, and 19-21 nucleotide residues. In some embodiments, one or more enzymes are included in the digestion assay such that the resulting RNA sequence fragments are short oligonucleotides having a length between 2-6, 6-10, and 10-14 nucleotide residues. In some embodiments, one or more enzymes are included in the digestion assay such that the resulting RNA sequence fragments are short oligonucleotides having a length between 2-6, 7-12, and 13-20 nucleotide residues. In some embodiments, the digestion assay comprises one or more enzymes in a concentration corresponding with the concentration and length of RNA sequences to be digested. In other words, the digestion assay may be adapted to provide a sufficient concentration of enzyme to cleave the RNA sequences into desired lengths for analyzing with a LC-MS system according to the concentration and length of the RNA sequences to be tested.

In some embodiments, the digestion assay comprises one or more enzyme derived from an organisms, including but not limited to animals (e.g., mammals, humans, cats, dogs, cows, horses, etc.), bacteria (e.g., E. coli, S. aureus, Clostridium spp., etc.), and mold (e.g., Aspergillus oryzae, Aspergillus niger, Dictyostelium discoideum, etc.). In some embodiments, the digestion assay comprises one or more enzyme which is recombinantly produced. For example, a gene encoding an RNase enzyme from one species (e.g., RNase T1 from A. oryzae) can be expressed in a bacterial host cell (e.g., E. coli) and purified. In some embodiments, the digestion is performed by an A. oryzae RNase T1 enzyme.

In some embodiments, the digestion assay comprises the enzyme ribonuclease T1 (RNase T1) which cleaves a single-stranded RNA sequence after a “G” motif (i.e., a guanosine residue). In some embodiments, the digestion assay comprises the enzyme ribonuclease colicin E5 (RNase colicin E5) which cleaves a single-stranded RNA sequence after a “GU” motif (i.e., a guanine residue followed by an uracil residue). In some embodiments, the digestion assay comprises the enzyme ribonuclease MCI (RNase MC1) which cleaves a single-stranded RNA sequence between a “C_U” motif (i.e., between a cytosine residue and an uracil residue), between an “A_U” motif (i.e., between an adenine residue and an uracil residue), between an “U_U” motif (i.e., between a first uracil residue and a second uracil residue), and between a “C_A” motif (i.e., between a cytosine residue and an adenine residue). In some embodiments, a digestion assay may include an enzyme which has a stronger preference for cleaving one or more first motifs over one or more second motifs (e.g., where enzyme has a higher affinity to one or more motifs and over a defined period of time the enzyme will cleave a higher number of the preference higher affinity motifs). For example, RNase MC1 has stronger preference for the “C_U” and “A_U” motifs than the “U_U” and “C_A” motifs. In some embodiments, the digestion assay comprises the enzyme ribonuclease cusativin (RNase cusativin) which cleaves between a C_U motif, a C_A motif, a “G_G” motif (i.e., between a first guanosine residue and a second guanosine residue), a U_U motif, and an “U_A” motif (i.e., between an uracil residue and an adenine residue), wherein the RNase cusativin has a stronger preference for the C_U, C_A, and the G_G motifs than the U_U and U_A motifs.

In some embodiments, the digestion assay comprises one or more ribonuclease enzymes selected from a group consisting of RNase colicin E5, RNase cusativin, RNase MC1, RNase T1, or any combination of two or more thereof. In some embodiments, RNase T1 is used to determine the identity (i.e., sequence mapping) of an RNA sequence. In some embodiments, RNase colicin E5 is used to determine the identity of an RNA sequence. In some embodiments RNase cusativin is used to determine the identity of an RNA sequence. In some embodiments RNase MC1 is used to determine the identity of a test mRNA. In some embodiments, RNase T1 is used in combination with another enzyme which is not RNase T1 to determine the identity of an RNA sequence.

In some embodiments, the digestion assay comprises at least one of RNase enzyme selected from a group comprising prokaryotic endonuclease enzymes (e.g., MazF, RecBCD endonuclease, T7 endonuclease, T4 endonuclease, Bal 31 endonuclease, micrococcal nuclease, etc.), tRNAse-type nuclease enzymes (e.g., RNase colicin E5, colicin D, PrrC, etc.), and eukaryotic nuclease enzymes (e.g., Neospora endonuclease, S1-nuclease, P1-nuclease, mung bean nuclease 1, Ustilago nuclease, Endo R, etc.).

In some embodiments, the digestion assay comprises one or more ribonuclease enzymes selected from a group consisting of exoribonuclease I, exoribonuclease II, oligoribonuclease, polynucleotide phosphorylase (PNPase), RNase A, RNase colicin E5, RNase cusativin, RNase D, RNase E, RNase H, RNase L, RNase MC1, RNase P, RNase PH, RNase PhyM, RNase R, RNase T, RNase T1, RNase T2, RNase U2, RNase V, and RNase III, or any combination of two or more thereof.

In some embodiments, the digestion assay comprises one or more enzymes selected from a group comprising Bal 31 endonuclease, colicin D, Endo R, eukaryotic nuclease enzymes, exoribonuclease I, exoribonuclease II, MazF, micrococcal nuclease, mung bean nuclease 1, Neospora endonuclease, oligoribonuclease, P1-nuclease, polynucleotide phosphorylase (PNPase), prokaryotic endonuclease enzymes, PrrC, RNase A, RNase colicin E5, RNase cusativin, RNase D, RNase E, RNase enzymes, RNase H, RNase L, RNase MC1, RNase P, RNase PH, RNase PhyM, RNase R, RNase T, RNase T1, RNase T2, RNase U2, RNase V, RNase III, S1-nuclease, tRNAse-type nuclease enzymes, T4 endonuclease, T7 endonuclease, Ustilago nuclease, or any combination of two or more thereof.

### Protecting Primers

In some embodiments, protecting primers are configured to hybridize with a portion of an RNA molecule. In some embodiments, the primer hybridized with the RNA molecule is combined with a digestion assay comprising an enzyme capable of cleaving an unprotected region of the RNA molecule so as to form two or more RNA fragments. For example, the digestion assay may comprise an enzyme capable of cleaving the RNA molecule in an unhybridized region of the RNA molecule while the enzyme is incapable of cleaving the RNA molecule in a region hybridized with the primer. In some embodiments, a digestion assay having a selective enzyme such as RNase T1 is beneficial for controlling the length of RNA fragments because the enzyme typically is incapable of cleaving the sterically form folded RNA. Therefore, the enzyme only cleaves the RNA molecule after G-bases in the unhybridized region of the RNA molecule and cleaves the RNA molecule only when the G-base is not base paired with the primer (i.e., protected from the enzyme).

In some embodiments, the primer is functionalized with a retentive tag. In some embodiments, the retentive tag is a C6 linker at 3′ end. In some embodiments, the retentive tag is a C12 linker at 3′ end. In some embodiments, the retentive tag is a C18 linker at 3′ end. In some embodiments, the highly retentive tag comprises an amino group, a thiol group, biotin, a florescent tag, a chromophore, an ionic tag, a spacer arm, or a combination of any two or more thereof. In some embodiments, the highly retentive tag is a C6 and/or a C18 alkyl linker. Alkyl linkers such as C6 and C18 may increase the hydrophobicity of the primer and therefore increase retention time in the column, allowing the primers to elute outside the elution window of the RNA fragments.

In some embodiments, the highly retentive tag is configured to enhance the retention of the primer in a liquid chromatography column. In some embodiments, the highly retentive tag is configured to modify the primer such that the primer elutes from a liquid chromatography column at a rate faster or slower than the rate of elution of the RNA fragments. The highly retentive tag may help in the separation and analysis of RNA fragment sequences by separating the elution time of the RNA fragments from the primer based on properties such as size, sequence, and/or structure. In some embodiments, the primer is functionalized with a highly retentive tag within three nucleotides of a first end. In some embodiments, the primer is functionalized with a highly retentive tag within 3 nucleotides of a second end. In some embodiments, the RNA protected region 306 is functionalized with a highly retentive tag within three nucleotides of the first end, the second end, or both the first and second end. In some embodiments, the highly retentive tag is selected to have properties which are distinct from tested hybrid in a LC column. In some embodiments, the highly retentive tag is hydrophobic so as to retain more in a reversed phase LC. In some embodiments, the highly retentive tag is hydrophilic, so it is retained in hydrophilic interaction chromatography (HILIC) mode. In some embodiments, the highly retentive tag is charged so as it is retained at a different rate than the test sequences in ion-exchange LC.

In some embodiments, the primer comprises a 2′O-methylated DNA. In some embodiments, the primer comprises a synthetic DNA oligonucleotide, a PNA, a morpholino DNA, 2′O-methylated DNA, or a combination of two or more thereof. In some embodiments, the primer is selected to optimize for high stability RNA/primer complexes and/or high melting temperatures known to those skilled in the art.

### Synthetic DNA Oligonucleotide Primers

In some embodiments, the primer comprises a synthetic DNA oligonucleotide having a length between 10-20 nucleotides. In some embodiments, the synthetic DNA oligonucleotides is configured to have a complementary sequence with a portion of an RNA molecule to hybridize with the RNA molecule and form RNA/DNA hybrids. In some embodiments, the hybrids are RNA/DNA, RNA/RNA, or peptide nucleic acid (PNA)/RNA hybrids. In some embodiment, a mixture of different primers is used to form a mixture of two or more hybrids selected from the group consisting of RNA/DNA, RNA/RNA, and PNA/RNA hybrids. In some embodiments, the length of the synthetic DNA oligonucleotide primer is selected to maximize the strength and stability of the DNA/RNA hybrids. For example, short primers may have insufficient strength and stability when hybridizing with the RNA molecule to remain hybridized during digestion which would result in incomplete digestion inhibition by the primer.

Since digestion assays comprising nucleases such as RNase T1 are specific to single stranded RNA, they will not cleave the protected regions of the RNA molecules hybridized with the primer. In some embodiments, specific RNA regions will not be digested despite including a motif recognized by an enzyme in the digestion assay for chevage. For example, in a digestion assay including RNase T1, cleavage of a recognition cleavage site such as the motif x/G may be inhibited by selecting primers to hybridize with the RNA molecule that block the positions with multiple G's or G spaced very closely in the sequence (e.g. GGG, GGAGCGGCC . . . , etc Preventing cleavage of repetitive motif such as these multiple G's or G spaced very closely in the sequence may be beneficial to prevent digestion of the RNA molecule into single G clips or very short fragments which can be unstable and difficult to detect in RNA mapping.

In some embodiments, one or several synthetic DNA complementary sequences are configured to be hybridized with an equimolar ratio with the target RNA oligonucleotide. In some embodiments, one or several DNA complementary sequences are configured to be hybridized in molar excess with the target RNA oligonucleotide. In some embodiments, one or several DNA complementary sequences are configured to be hybridized with a sub-equimolar ratio with the target RNA oligonucleotide.

In some embodiments, a digestion assay is combined with a target RNA oligonucleotide and a primer, wherein the target RNA oligonucleotide and the primer are added at sub-equimolar ratios relative to each other. For example, adding a target RNA oligonucleotide and a primer at sub-equimolar ratios may result in a first portion of the RNA molecules being completely digested by an enzyme in the digestion assay, while a second portion of the RNA molecule are protected and therefore result in longer RNA oligonucleotide fragments. In some embodiments, 100% of the RNA sequence is mapped by assembling all long and short detected RNA fragments from the digestion assay.

### Morpholino DNA Primers

In some embodiments, the primer comprises morpholino DNA. Morpholino DNA contains the same nucleic acid base pairs as standard DNA molecules and is therefore capable of hybridizing with and being complementary to a portion of an RNA molecule. Additionally, morpholino DNA comprise nucleic acid bases which are bound to methylenemorpholine rings linked through phosphorodiamidate moieties instead of phosphate moieties. As the phosphorodiamidate groups are uncharged, compared to the anionic phosphates of standard DNA, the negative ionization within a physiological pH range of standard DNA molecules is eliminated. In some embodiments, the morpholino DNA primers are between 10-25 nucleotides in length.

### PNA Primers

In some embodiments, the primer comprises a peptide nucleic acid (PNA). PNA contains the same nucleic acid base pairs as standard DNA molecules and is therefore capable of hybridizing with and being complementary to a portion of an RNA molecule. Additionally, PNA has a backbone of repeating N-(2-aminoethyl)glycine units, to which the nucleobases are attached with a methylene carbonyl linker instead of phosphate moieties. The structure of PNA may provide any one or more of a higher binding affinity for PNA/RNA hybrids compared to DNA/RNA hybrids due to the lack of charge repulsion between PNA and the target nucleic acids, a higher resistance to enzymatic degradation, faster hybridization kinetics, and a higher specificity for the RNA molecule. In some embodiments, the PNA primers are between 10-25 nucleotides in length.

### Optimizing Digestion Conditions

In some embodiments, the conditions for hybridizing the RNA molecule with a primer either separately or together with a digestion assay are selected to maximize the strength and stability of the primer/RNA hybrids. In some embodiments, the primer selection, the digestion temperature, and the buffer and/or cations are optimized to enhance stability of the primer/RNA hybrids.

### Primer Selection

In some embodiments, the length of the primer is selected to maximize the strength and stability of the primer/RNA hybrids. For example, primers which are too short may result insufficient strength and stability to remain continuously hybridized with the RNA molecule, rending the digestion inhibition by the primer incomplete In some embodiments, the type of primer is selected to maximize stability of the hybrid. For example, the primer may comprise morpholino DNA and/or PNA resulting in hybrids having higher melting temperatures and stability relative to DNA/RNA hybrids. In some embodiments, primer/RNA hybrids with higher melting temperatures are more stable and therefore require fewer hybridization stability enhancements.

### Digestion Temperature

The optimal temperature to hybridize a primer with an RNA molecule may vary depending on enzyme specificity, enzyme concentration relative to RNA concentration, the length and structure of the RNA molecule, and pH of the solution. For example, the optimal temperature for enzymatic digestion of primer/RNA hybrids may be about 37° C. However, at this optimal digestion temperature primer/RNA hybrids may be less stable leading to reduced protection of the RNA molecule by the primer. Reduced primer effectiveness may result in shorter and more RNA fragments during digestion therefore adding complication to mapping the sequence of the RNA molecule. Therefore, the optimal temperature may be selected based on the enzyme kinetics for digestion and the stability of the primer/RNA hybrids.

In some embodiments, digestion of the RNA molecule is carried out at any temperature at which the enzyme will perform its intended function of digesting the RNA molecule in a given timeframe. In some embodiments, the temperature is 37° C. In some embodiments, the temperature is between 15-24° C. In some embodiments, the temperature is between 20-100° C. In some embodiments, the temperature is between 30-50° C. In some embodiments, the temperature is between 10-20° C., 20-30° C., 30-40° C., 40-50° C., 50-60° C., 60-70° C., 70-80° C., 80-90° C., or 90-100° C.

In some embodiments, the timeframe for digestion is between 0.5-1 seconds, 1-60 seconds, 1-5 minutes, 5-10 minutes, 10-20 minutes, 20-30 minutes, 30-40 minutes, 40-50 minutes, or 50-60 minutes. In some embodiments, the timeframe for digestion is between 1-2 hours, 2-3 hours, 3-4 hours, 4-5 hours, 5-6 hours, 6-7 hours, 7-8 hours, 8-9 hours, 9-10 hours, 10-11 hours, 11-12 hours, 12-13 hours, 13-14 hours, 14-15 hours, 15-16 hours, 16-17 hours, 17-18 hours, 18-19 hours, 19-20 hours, 20-21 hours, 21-22 hours, 22-23 hours, or 23-24 hours. In some embodiments, the timeframe for digestion is between 24-25 hours, 25-26 hours, 26-27 hours, 27-28 hours, 28-29 hours, 29-30 hours, 30-31 hours, 31-32 hours, 32-33 hours, 33-34 hours, 34-35 hours, 35-36 hours, 36-37 hours, 37-38 hours, 38-39 hours, 39-40 hours, 40-41 hours, 41-42 hours, 42-43 hours, 43-44 hours, 44-45 hours, 45-46 hours, 46-47 hours, or 47-48 hours.

### Buffers and Cations

In some embodiments, buffers are added to the digestion assay and/or the mixture of primer and RNA molecules to improve primer/RNA hybrid stability relative to digestion with no buffer. In some embodiments, the digestion is performed in a buffer solution. In some embodiments, the buffer is configured to maintain a pH within the digestion assay between 6-10 pH. In some embodiments, the buffer is configured to maintain a pH within the digestion assay between 6.5-7, 7-7.5, 7.5-8, 8.5-9, or 9.5-10 pH. In some embodiments, the concentration of each buffering agent in a buffer solution ranges from 1-200 mM. In some embodiments, the concentration of each buffering agent in a buffer solution ranges from 1-20 mM, 10-50 mM, 25-100 mM, or 75-200 mM.

In some embodiments, the buffer solution comprises ammonium acetate (AmAc), triethylamine acetate (TEAAc), hexylamine acetate (HAAc), diisopropyl-ethylamine acetate (DIPEAAc), hexafluoropropanol (HFIP), sodium phosphate buffer (NaPhos), or any combination of two or more thereof. In some embodiments, the buffer solution comprises ammonium acetate (AmAc), HEPES (4-2-hydroxyethyl-1-piperazineethanesulfonic acid), hexafluoropropanol (HFIP), hexylamine acetate (HAAc), MOPS (3-(N-morpholino) propanesulfonic acid), PIPES (piperazine-N,N′-bis(2-ethanesulfonic acid)), sodium bicarbonate, Sodium phosphate (NaPhos), Triethylammonium acetate (TEAAc), Tris-acetate, Tris-base, Tris-Cl, and DBAA, diisopropyl-ethylamine acetate (DIPEAAc), or any combination of two or more thereof.

In some embodiments, the small cations are added to the buffer solution to further promote hybridization of the primer with the RNA molecule relative to solutions with no small cations. For example, the small cations may increase the melting temperature of an RNA/primer hybrid by increasing the ionic strength of the hybridizing interaction. In some embodiments, the small cations are configured to prevent one or both ends of the primer from separating from the RNA molecule. In some embodiments, the small cations comprise magnesium (Mg²⁺). In some embodiments, the small cations comprise magnesium (Mg²⁺) and/or sodium (Na²⁺). In some embodiments, the small cations are added to the buffer at a concentration between 5-25 mM. In some embodiments, the small cations are added to the buffer at a concentration between 5-10, 10-15, 15-20, or 20-25 mM. In some embodiments, the small cations within the buffer solution are at a concentration between 5-25 mM. In some embodiments, the concentration of buffer is 75 mM. In some embodiments, the concentration of buffer is above 75 mM. In some embodiments, the concentration of magnesium cations is between 5 and 25 mM.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention.

### Example 1: RNA Mapping with RNase T1 Endonuclease

Traditional methods for mapping RNA sequences are challenging to for sequences that are rich in guanine (G). RNA sequences that contain motifs like GG, GGG, GGCGGGCGC, etc. are digested into short RNA fragments such as mononucleotides and dinucleotides, which are difficult to detect and provide limited information on the original parent RNA sequence with LC-MS as discussed above.

Sequence mapping of RNA such as mRNA using LC-MS typically achieves 30-50% sequence coverage. Sequence coverage may be improved by conducting LC-MS/MS sequencing to disambiguate two to six nucleotide long RNA fragments of identical mass. In some embodiments, to achieve full RNA sequence coverage, a digestion assay comprising a plurality of “orthogonal” enzymes is used.

In some embodiments, partial digestion is used to achieve higher sequence coverage for endonuclease enzymes like RNase T1 by allowing for missed-cleaved G sites within the RNA sequence. However, partial digestion may be challenging to control as RNase T1 does not have an efficient inhibitor that can be employed to stop the digestion at the optimal moment. Partial digestion may also create cyclic phosphate termini on the RNA oligonucleotides in addition to the regular phosphate termini, splitting the RNA oligonucleotide chromatogram signals into two peaks.

Partial digestion was conducted using an exemplary 50 nucleotide length RNA molecule having the sequence AGACAGUUUCGACUGGAUACACCUUGAUGUUAAACGUCCUACCUCCGCCA (SEQ ID NO: 1). The RNA molecule was digested with a digestion assay comprising an RNAse T1 endonuclease to cleave the RNA molecule into RNA fragments at a temperature of 25° C. The RNA molecule was digested with RNase T1 for four minutes and for ninety minutes. Upon completion of digestion the RNA fragments were analyzed using LC-MS. Since RNase T1 cleaves the RNA sequences at every G position within the RNA molecules, cleavage of the RNA molecule could result in between 2-9 fragments. As such, the exemplary RNA sequence described herein is capable of being cleaved by RNase T1 at the 2^{nd}, 6^{th}, 11^{th}, 15^{th}, 16^{th}, 26^{th}, 29^{th}, 36^{th}, and 47^{th} positions.

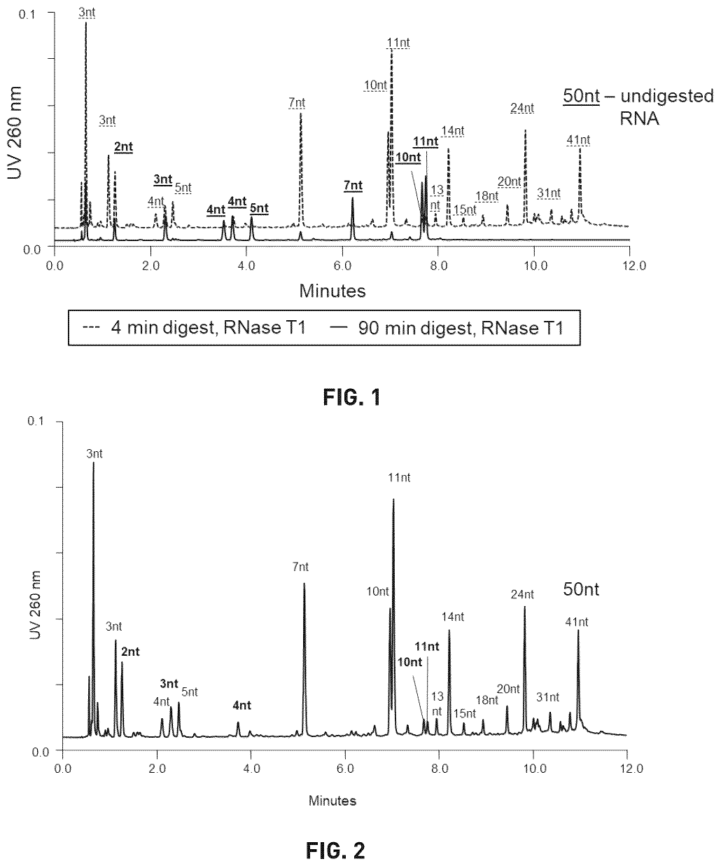

FIG. 1 illustrates an example of a liquid chromatography elution chromatogram for partial digestion of an RNA molecule after four minutes of digestion and after ninety minutes of digestion. The solid line represents elution of fragments from the ninety minute digestion. The dashed line and corresponding underlined nucleotide base pair labels represent the digestion after four minutes. The fewer fragments shown from the four minute digestion compared with the ninety-minute digestion illustrates that a partial digestion at four minutes has multiple miss-cleaved oligonucleotides, such as with cyclic phosphate on 3′-end of the RNA sequence. Analysis of the chromatogram illustrates a significant increase in larger cleavage products in the sample digested for four minutes compared to the sample digested for 90 minutes. The disappearance of many of the late-eluting peaks between the 4 minute and 90 minute digestion samples indicates that the RNA sequence is fully cleaved after 90 minutes.

FIG. 2 illustrates the four minute partial digestion shown in FIG. 1, decoupled from the ninety minute partial digestion chromatogram. The cleavage products were determined by high performance liquid chromatography of the resultant mixture. Table 1 shows the RNA fragment products created from the digestion. RNA fragments denoted in bold font represent those that are completely digested and have a phosphate terminus, abbreviated p. Oligonucleotides denoted in regular font are those that have cyclic phosphate termini, abbreviated pc.

TABLE 1

| Peak label | Sequence |
|---|---|
| 3 nt | CCAp |
| 2 nt | AGp |
| 3 nt | AUGpc |
| 3 nt | AUGp |
| 4 nt | ACUGpc |
| 4 nt | ACAGp |
| 5 nt | UUUGCpc |
| 7 nt | UUAAACGpc |
| 10 nt | AUACACCUUGpc (SEQ ID NO: 2) |
| 11 nt | UCCUACCUCCGpc (SEQ ID NO: 3) |
| 10 nt | UCCUACCUCCGp (SEQ ID NO: 3) |
| 11 nt | AUACACCUUGp (SEQ ID NO: 2) |
| 13 nt | ACAGUUUCGACUGpc (SEQ ID NO: 4) |
| 14 nt | ACAGUUUCGACUGGpc (SEQ ID NO: 5) |
| 15 nt | ACUGGAUACACCUUGpc (SEQ ID NO: 6) |
| 18 nt | UUAAACGUCCUACCUCCGpc (SEQ ID NO: 7) |
| 20 nt | AUACACCUUGAUGUUAAACGpc (SEQ ID NO: 8) |
| 24 nt | ACAGUUUCGACUGGAUACACCUUGpc (SEQ ID NO: 9) |
| 31 nt | AUACACCUUGAUGUUAAACGUCCUACCUCCGpc (SEQ ID NO: 10) |
| 41 nt | UUUCGACUGGAUACACCUUGAUGUUAAACGUCCUACCUCCGpc |
| | (SEQ ID NO: 11) |
| 50 nt | AGACAGUUUCGACUGGAUACACCUUGAUGUUAAACGUCCUACCC |
| | CGCCA (SEQ ID NO: 12) |

As illustrated in table 1, full sequence coverage was obtained with a partial digestion strategy. In some cases, the miss-cleaved oligonucleotides created in a partial digestion strategy are rapidly converted into shorter cleavage products because it is difficult to stop the digestion prevent the desirable miss-cleaved product from disappearing.

### Example 2: Measuring Melting Temperature of RNA/Primer Hybrids

The addition of a primer such as a complementary DNA oligonucleotides to an RNA molecule to form an RNA/primer hybrid to protect regions of the RNA molecule from enzymatic digestion, such as RNase T1. However, the RNA/primer hybrid may be unstable when too short primers are used. Short primers such as those comprising complimentary DNA oligonucleotides have low melting temperatures, which indicates low thermal stability. Synthesizing longer, more stable, complementary DNA oligonucleotides is expensive and impractical in many applications. In some embodiments, digestion conditions are optimized to overcome the challenges associated with using DNA primers as discussed above. In some embodiments, low temperatures for digestions are used to provide stability to the RNA/primer hybrid. In some embodiments, the temperature for digestion is between 10-25° C. In some embodiments, buffers with high ionic concentrations are used to improve the melting temperature of DNA complimentary oligonucleotides. In some embodiments, DNA with high melting point and high hybridization stability is used as the primer. In some embodiments, a primer with high stability such as morpholino DNA and/or PNA is used in the primer.

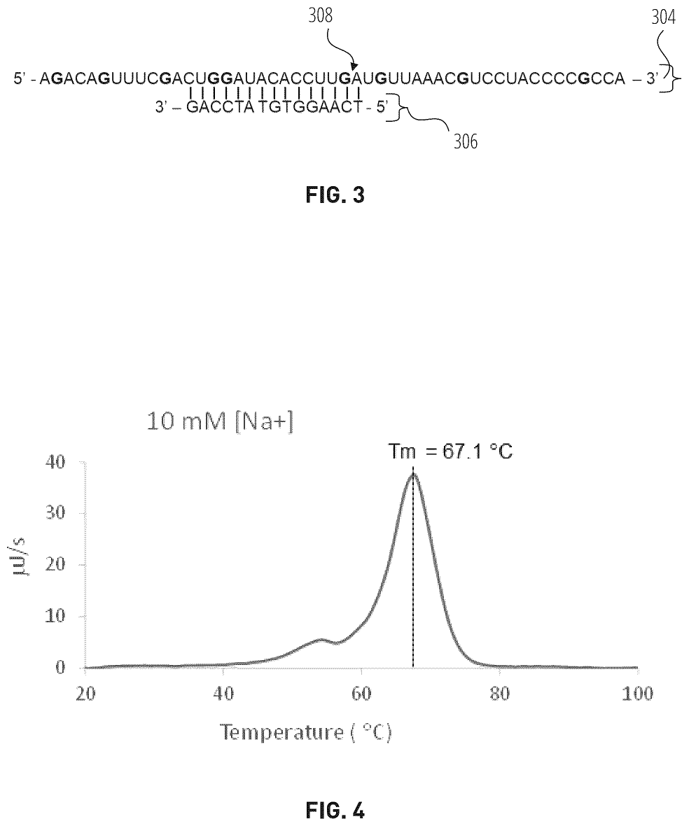

FIG. 3 illustrates a 50 nucleotide RNA molecule 304 hybridized with a primer. In some embodiments, the location of the primer along the 50 nucleotide RNA molecule 304 creates an RNA protected region 306 and blocks endonuclease enzyme cleavage sites. In some embodiments, the number of primer protected cleavage sites on the RNA molecule is three. In some embodiments, the primer has a length between 15-20 nucleotides. In some embodiments, the primer extends from a first end to a second end. In some embodiments, the primer hybridizes with a complementary portion of the 50 nucleotide RNA molecule 304 from the first end to the second end. In some embodiments, two or three nucleotides of the first end of the primer are configured to transition between being attached to and unattached from the complementary portion of the 50 nucleotide RNA molecule 304. In some embodiments, two or three nucleotides of the second end of the primer are configured to transition between being attached to and unattached from the complementary portion of the 50 nucleotide RNA molecule 304. In some embodiments, the cleavage motifs near the terminal duplex positions are still accessible to enzymatic digestion, due to dynamic partial opening and closing of duplex ends. For example, RNase T1 may cleave cleavage site 308 when an end of the primer is unattached from the 50 nucleotide RNA molecule 304.

In some embodiments, the melting temperature (Tₘ) of the RNA/primer hybrid in a 50 mM buffer solution is 49.4° C. In some embodiments, the Tₘ of the RNA/primer hybrid in a 50 mM buffer solution is between 45-55° C. In some embodiments, the primer is selected to provide the hybridized molecule with a Tₘ between 60-70° C. In some embodiments, partial separation of the RNA/primer hybrid results in partial cleavage of the 50 nucleotide RNA molecule 304. In some embodiments, the GG position along the 50 nucleotide RNA molecule 304 is completely protected by the RNA protected region 306. In some embodiments, no digestion of the GG site along the 50 nucleotide RNA molecule 304 is observed. In some embodiments, partial digestion of the cleavage site 308 near the terminus of the RNA protected region 306 is observed. For example, the partial digestion of the cleavage site 308 could be due to partial opening of the RNA/primer hybrid at the terminus of the RNA protected region 306, rendering the G positions near the termini accessible to the endonuclease enzyme RNase T1.

FIG. 4 illustrates an example of a nano-differential scanning calorimetry (DSC) thermogram used to measure the melting temperature (Tₘ) for a 21/23 nucleotide RNA duplex for the buffer conditions of 10 mM [Na+]. For example, Tₘ is measured as a max of differential scanning calorimetry curve. As shown in FIG. 4, for example, the wide peak at base indicates that a partial melting occurs at temperatures below Tₘ. In some embodiments, partial termini melting/opening may occur at 10-15° C. below the measured Tₘ.

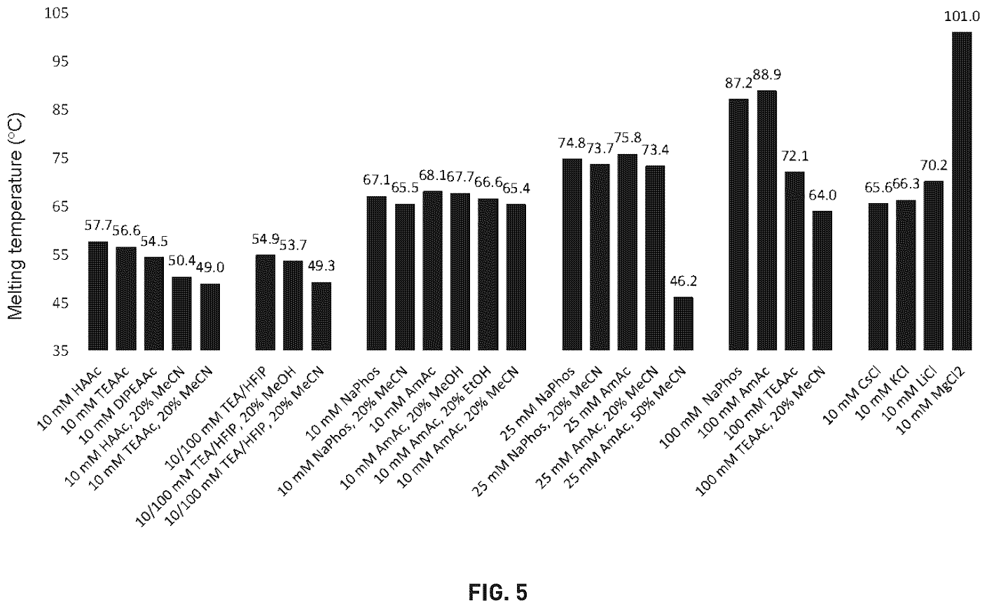

FIG. 5 illustrates the melting temperature of 21/23 nucleotide RNA/primer hybrids in a plurality of buffer solutions. The following abbreviations are used in FIG. 5: AmAc—ammonium acetate, TEA—triethylamine, HA—hexylamine, DIPEA—diisopropyl—ethylamine, HFIP—hexafluoropropanol, MeOH—methanol, EtOH—ethanol, MeCN—acetonitrile, NaPhos—sodium phosphate buffer. As the concentration of the buffer solution was increased, the melting point of the RNA/primer hybrids increased as well. As shown in FIG. 5, for example, the melting point for the RNA/primer hybrids held in a buffer solution of 10 mM NaPhos was measured at 67.1° C., while the melting point for the RNA/primer hybrid held in a buffer solution of the 100 mM NaPhos was measured at 87.2° C. This same increase in melting temperature with higher concentrations of buffer solution was also observed for RNA/primer hybrids in 10 mM AmAc and 100 mM AmAc buffer solutions.

In some embodiments, the addition of small cations is used to improve the melting temperature of primer/RNA duplexes. In some embodiments, the small cations are magnesium ions (Mg²⁺). As shown in FIG. 5, for example, the melting point of the primer/RNA duplexes reached a maximum of 101.0° C. when in a 10 mM solution of MgCl₂. In solution, the MgCl₂ dissociates into the ions, Mg²⁺ and Cl. This demonstrates the ability of Mg²⁺ to efficiently stabilize RNA duplexes.

### Example 3:50 nt RNA Molecule Digestion with RNase T1 and a Primer

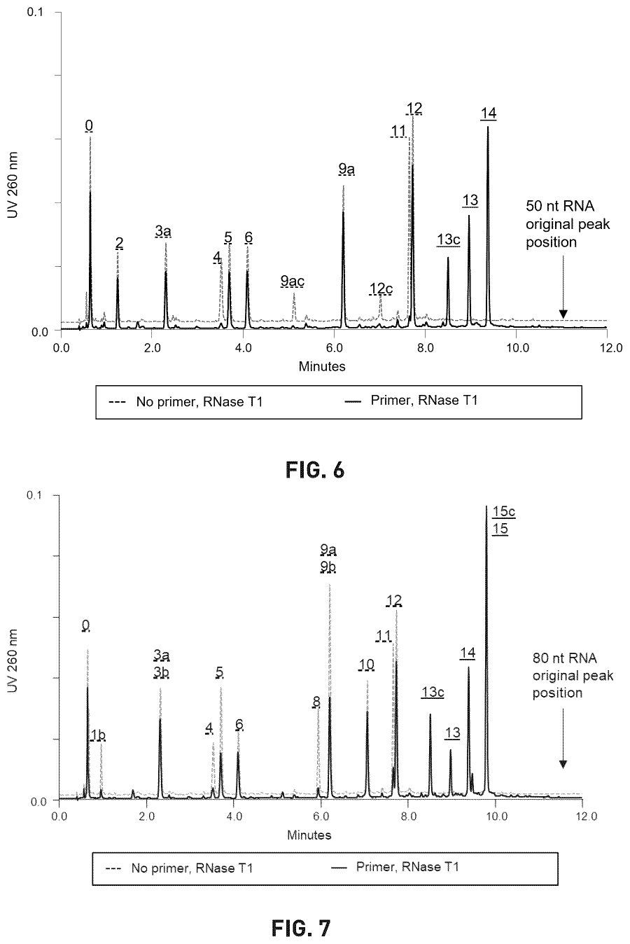

FIG. 6 illustrates an example of a high performance liquid chromatography elution of RNA fragments from an exemplary RNA molecule. The exemplary RNA molecule has the following 50 nucleotide long sequence: AGACAGUUUCGACUGGAUACACCUUGAUGUUAAACGUCCUACCUCCGCCA (SEQ ID NO: 1). The RNA molecule was combined with a protecting DNA complementary oligonucleotide primer to from an RNA/primer hybrid protecting at least a portion of the RNA molecule from enzymatic degradation. The RNA/primer hybrid was digested with the RNase T1 endonuclease enzyme. The RNase T1 cleaved the exemplary RNA molecule into RNA fragments which were sequenced using LC-MS. Table 2 shows the RNA oligonucleotide cleavage products created from the digestion. Oligonucleotides denoted in bold font represent those that new oligonucleotides observed only in the sample that included the protecting DNA complementary oligonucleotide primer.

TABLE 2

| Peak label | Sequence |
|---|---|
| 0 | CCA |
| 2 | AGp |
| 3a | AUGp |
| 4 | ACUGp |
| 5 | ACAGp |
| 6 | UUUCGp |
| 9ac | UUAAACGpc |
| 9a | UUAAACGp |
| 12c | UCCUACCUCCGpc (SEQ ID NO: 3) |
| 11 | AUACACCUUGp (SEQ ID NO: 2) |
| 12 | UCCUACCUCCGp (SEQ ID NO: 3) |
| 13c | ACUGGAUACACCUUGpc (SEQ ID NO: 6) |
| 13 | ACUGGAUACACCUUGp (SEQ ID NO: 6) |
| 14 | Primer |

As shown in FIG. 6, for example, the disappearance of peaks 4 and 11 in the chromatogram of the sample with the protected DNA complementary oligonucleotide primer indicate that the cleavage sites at 5′-end of the RNA sequence are well protected by the DNA complementary oligonucleotide. The presence of the peak 3a in both chromatograms indicates 3′-blocked terminus may not completely stop the digestion of the RNA molecule. The presence of peak 13 and 13c in the chromatogram of the sample with the primers indicates a new RNA fragment was created by the protecting the RNA molecule with the primer.

### Example 4:80 nt RNA Molecule Digestion with RNase T1 and a Primer

FIG. 7 illustrates an example of a high performance liquid chromatography elution of RNA fragments from an exemplary RNA molecule, according to embodiments described herein. An 80 nucleotide exemplary RNA molecule was used having the sequence: ACGUAAACUGCGGACAGUUACUCGAUUCAAAGACAGUUUCGACUGGAUACACCUU GAUGUUAAACGUCCUACCUCCGCCA (SEQ ID NO: 14).

The RNA molecule was combined with a first primer and a second primer, where each primer is a protecting DNA complementary oligonucleotide, to from an RNA/primer hybrid protecting at least a first portion of the RNA molecule with the first primer and a second portion of the RNA molecule spaced apart from the first portion with the second primer from enzymatic degradation (See FIG. 9). The RNA/primer hybrid was digested with an RNAse T1 endonuclease enzyme. The RNase T1 cleaved the exemplary RNA molecule into RNA fragments which were sequenced using LC-MS. Table 3 shows the sequences of RNA fragments determined by LC-MS.

TABLE 3

| Peak label | Sequence |
|---|---|
| 0 | CCA |
| 1b | CGp |
| 3a | AUGp |
| 3b | ACGp |
| 4 | ACUGp |
| 5 | ACAGp-two fragments |
| 6 | UUUCGp |
| 8 | UUACUCGp |
| 9a | UUAAACGp |
| 9b | UAAACUGp-two fragments |
| 10 | AUUCAAAGp |
| 11 | AUACACCUUGp (SEQ ID NO: 2) |
| 12 | UCCUACCUCCGp (SEQ ID NO: 3) |
| 13c | ACUGGAUACACCUUGpc (SEQ ID NO: 6) |
| 13 | ACUGGAUACACCUUGp (SEQ ID NO: 6) |
| 14 | Second Primer |
| 15 | First Primer |
| 15c | UAAACUGCGGACAGUUACUCGpc |
| | (SEQ ID NO: 15) |

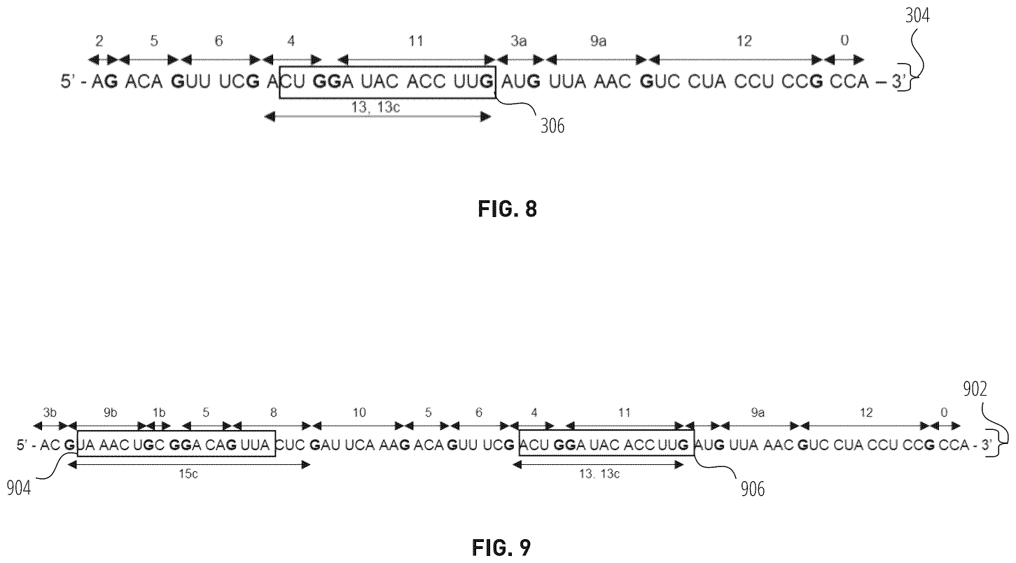

FIG. 8 depicts a schematic view of the RNA fragments within the 50 nucleotide RNA molecule 304 determined by LC-MS and shown in FIG. 6. The overlaying numerals represent the individual RNA fragments that are created as a result of endonuclease enzymatic digestion describe in Table 2. The RNA protected region 306 of the RNA molecule when hybridized with the primer protects the RNA molecule from an enzymatic chevage in the hybridized region. In some embodiments, longer RNA fragments are produced due protection from the primer (see peaks 13, 13c in Table 2). In some embodiments, shorter digestion products from within the RNA protected region 306 of the 50 nucleotide RNA molecule 304 are produced due RNA/primer hybrid instability at one or both ends of the protected region allowing for cleavage of the 50 nucleotide RNA molecule 304 near the ends of the region hybridized to the primer (see peaks 4, 11 in Table 2).

FIG. 9 depicts a schematic view of the RNA fragments within the exemplary 80 nt RNA molecule 902 hybridized to a first primer and a second primer as determined by LC-MS and shown in FIG. 7. In some embodiments, the area hybridized between the exemplary 80 nt RNA molecule 902 and the first primer create a first protected region 904 and the area hybridized between the exemplary 80 nt RNA molecule 902 and the second primer create a second protected region 906. In some embodiments, the second primer has a different sequence from the first primer with first protected region 904 spaced apart from the second protected region 906. In some embodiments, at least one of the first primer and the second primer is configured to protect a motif in the exemplary 80 nt RNA molecule 902 which would produce a mononucleotide, a dinucleotide, and/or a trinucleotide interacting with the digestion assay.

The first protected region 904 and the second protected region 906 block the endonuclease enzyme RNase T1 from cleaving the exemplary 80 nt RNA molecule 902 where the first primer and the second primer are hybridized to the exemplary 80 nt RNA molecule 902. In some embodiments, at least one of the first primer and the second primer are configured to hybridize with suspected mutation points within the exemplary 80 nt RNA molecule 902. In some embodiments, the exemplary 80 nt RNA molecule 902 comprises secondary and/or tertiary structures. In some embodiments, at least one of the first primer and the second primer are configured to linearize the exemplary 80 nt RNA molecule 902 when hybridized so as to increase the susceptibility of the exemplary 80 nt RNA molecule 902 to enzymes during digestion compared to the exemplary 80 nt RNA molecule 902 unhybridized to at least one of the first primer and the second primer. In some embodiments, longer digestion products are produced due to the presence the first protected region 904 and the second protected region 906 (see peaks 15, 13, 13c in Table 3). In some embodiments, shorter digestion products within the first protected region 904 and the second protected region 906 are produced due to RNA/primer hybrid instability at one or both ends allowing for cleavage of the exemplary 80 nt RNA molecule 902 within the first or second protected regions (see peaks 9b, 1b, 5, 8, 4, 11 in Table 3).

### Example 5: Assessment of Primers Functionalized with Retentive Tags

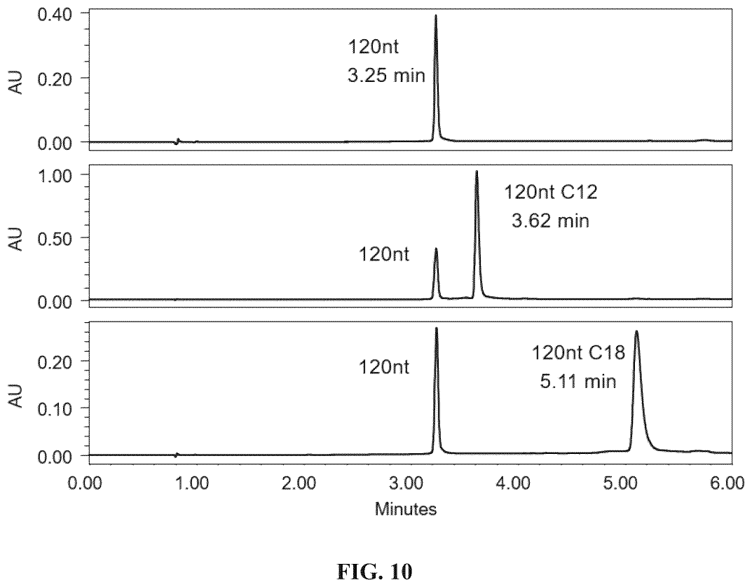

FIG. 10 illustrates an example of a high performance liquid chromatography elution of exemplary primers functionalized with retentive and highly retentive tags, according to embodiments described herein. Shown are exemplary deoxythymidine oligonucleotides of 120 nt either unlabeled (FIG. 10 top trace), labeled with a C12 dodecyl retentive tag (FIG. 10 middle trace), or labeled with a C18 (stearlyl) highly retentive tag (FIG. 10 bottom trace). The results demonstrate that the hydrophobic tags altered the retention of the tagged primers to later retention windows, which can help shift primers away from the digested oligonucleotides of interest and the original intact sgRNA molecule when the tagged primers are used as blocking primers in RNA/primer hybrid digestion assays using an RNAse T1 endonuclease enzyme, as described above.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

1. A method of mapping a sequence of an RNA molecule comprising:
hybridizing a primer with a portion of the RNA molecule to form an RNA/primer hybrid having a protected RNA region hybridized with the primer and an unprotected RNA region, wherein the primer is selected from a group consisting of a synthetic DNA oligonucleotide, a morpholino DNA, and a PNA;
digesting the RNA/primer hybrid in a digestion assay comprising an enzyme configured to cleave a motif within the unprotected RNA region of the RNA/primer hybrid thereby forming two or more RNA fragments; and
analyzing the two or more RNA fragments using liquid chromatography-mass spectrometry to determine a sequence of nucleotides in the RNA molecule.

2. The method of claim 1, wherein the primer comprises a sequence which is complementary with the protected RNA region and protects the protected RNA region from enzymatic cleavage.

3. The method of claim 1, wherein digesting is performed at a temperature between 15-24° C.

4. The method of claim 1, wherein the step of hybridizing the primer with the portion of the RNA molecule comprises combining the primer with the RNA molecule in equimolar ratios so as to provide complete protection of the RNA molecule from enzymatic digestion.

5. The method of claim 1, wherein the step of hybridizing the primer with the portion of the RNA molecule comprises combining the primer with the RNA molecule in sub-equimolar ratios so as to provide incomplete protection of the RNA molecule from enzymatic digestion.

6. The method of claim 1, wherein the primer has a length between 15-20 nucleotides.

7. The method of claim 1, wherein the primer is selected to provide an RNA/primer hybrid with a melting temperature between 60-70° C.

8. The method of claim 1, wherein the primer sequence extends from a first end to a second end, and wherein the primer hybridizes with a complementary portion of the RNA molecule from the first end to the second end, and wherein two or three nucleotides of the first end of the primer are configured to transition between being attached to and unattached from the complementary portion of the RNA molecule and wherein two or three nucleotides of the second end of the primer are configured to transition between being attached to and unattached from the complementary portion of the RNA molecule.

9. The method of claim 1, comprising hybridizing a second primer having a different sequence from the first primer with a second portion of the RNA molecule spaced apart from the portion of the RNA molecule hybridized with the first primer, wherein at least one of the first primer and the second primer is configured to protect a second motif in the RNA molecule which would produce a mononucleotide, a dinucleotide, and/or a trinucleotide interacting with the digestion assay.

10. The method of claim 1, wherein the primer is configured to hybridize with suspected mutation points within the RNA sequence

11. The method of claim 1, wherein the RNA molecule comprises secondary and/or tertiary structures, and wherein the primer is configured to linearize the RNA molecule when hybridized so as to increase the susceptibility of the RNA molecule to enzymes during digestion compared to the RNA molecule unhybridized to the primer.

12. The method of claim 1, comprising adding a buffer to the digestion assay, wherein the buffer is configured to stabilize the hybridizing of the primer with the RNA molecule relative to a digestion assay with no buffer, optionally wherein: (A) the buffer comprises ammonium acetate (AmAc), triethylamine acetate (TEAAc), hexylamine acetate (HAAc), diisopropyl-ethylamine acetate (DIPEAAc), hexafluoropropanol (HFIP), sodium phosphate buffer (NaPhos), or any combination of two or more thereof; or (B) the buffer solution comprises ammonium acetate (AmAc), HEPES (4-2-hydroxyethyl-1-piperazineethanesulfonic acid), hexafluoropropanol (HFIP), hexylamine acetate (HAAc), MOPS (3-(N-morpholino)propanesulfonic acid), PIPES (piperazine-N,N′-bis(2-ethanesulfonic acid)), sodium bicarbonate, Sodium phosphate (NaPhos), Triethylammonium acetate (TEA Ac), Tris-acetate, Tris-base, Tris-Cl, and DBAA, diisopropyl-ethylamine acetate (DIPEA Ac), or any combination of two or more thereof.

13. (canceled)

14. (canceled)

15. The method of claim 12, wherein the buffer has a concentration of at least 75 mM.

16. The method of claim 1, further comprising adding magnesium ions to the digestion assay at a concentration between 5-25 mM.

17. The method of claim 1, wherein the primer is functionalized with a highly retentive tag configured to modify the primer such that during liquid chromatography-mass spectrometry the primer elutes from a liquid chromatography column at a rate faster or slower than the rate of elution of the two or more RNA fragments.

18. The method of claim 17, wherein the primer is functionalized with the highly retentive tag within three nucleotides of a first end, within three nucleotides of a second end, or functionalized within three nucleotides of the first end and the second end, respectively.

19. The method of claim 1, wherein the primer is configured to protect 20-40% of the RNA molecule from enzymatic digestion.

20. The method of claim 1, wherein the digestion assay comprises RNase T1.

21. The method of claim 1, wherein the digestion assay comprises a ribonuclease enzyme selected from a group consisting of exoribonuclease I, exoribonuclease II, oligoribonuclease, polynucleotide phosphorylase (PNPase), RNase A, RNase Colicin E5, RNase cusativin, RNase D, RNase E, RNase H, RNase L, RNase M C1, RNase P, RNase PH, RNase PhyM, RNase R, RNase T, RNase T1, RNase T2, RNase U2, RNase V, and RNase III, or any combination of two or more thereof.

22. The method of claim 1, wherein the digestion assay comprises one or more enzymes selected from a group consisting of Bal 31 endonuclease, colicin D, Endo R, eukaryotic nuclease enzymes, exoribonuclease I, exoribonuclease II, MazF, micrococcal nuclease, mung bean nuclease 1, Neospora endonuclease, oligoribonuclease, P1-nuclease, polynucleotide phosphorylase (PNPase), prokaryotic endonuclease enzymes, PrrC, RNase A, RNase Colicin E5, RNase cusativin, RNase D, RNase E, RNase enzymes, RNase H, RNase L, RNase M C1, RNase P, RNase PH, RNase PhyM, RNase R, RNase T, RNase T1, RNase T2, RNase U2, RNase V, RNase III, S1-nuclease, tRNAse-type nuclease enzymes, T4 endonuclease, T7 endonuclease, Ustilago nuclease, or any combination of two or more thereof.


SEQUENCE LISTING (16 sequences)

SEQ ID NO: 1 - RNA, 50 nt, synthetic construct
  source 1..50: mol_type = other RNA
agacagtttcgactggatacaccttgatgttaaacgtcctacctccgcca

SEQ ID NO: 2 - RNA, 10 nt, synthetic construct
  source 1..10: mol_type = other RNA
atacaccttg

SEQ ID NO: 3 - RNA, 11 nt, synthetic construct
  source 1..11: mol_type = other RNA
tcctacctccg

SEQ ID NO: 4 - RNA, 13 nt, synthetic construct
  source 1..13: mol_type = other RNA
acagtttcgactg

SEQ ID NO: 5 - RNA, 14 nt, synthetic construct
  source 1..14: mol_type = other RNA
acagtttcgactgg

SEQ ID NO: 6 - RNA, 15 nt, synthetic construct
  source 1..15: mol_type = other RNA
actggatacaccttg

SEQ ID NO: 7 - RNA, 18 nt, synthetic construct
  source 1..18: mol_type = other RNA
ttaaacgtcctacctccg

SEQ ID NO: 8 - RNA, 20 nt, synthetic construct
  source 1..20: mol_type = other RNA
atacaccttgatgttaaacg

SEQ ID NO: 9 - RNA, 24 nt, synthetic construct
  source 1..24: mol_type = other RNA
acagtttcgactggatacaccttg

SEQ ID NO: 10 - RNA, 31 nt, synthetic construct
  source 1..31: mol_type = other RNA
atacaccttgatgttaaacgtcctacctccg

SEQ ID NO: 11 - RNA, 41 nt, synthetic construct
  source 1..41: mol_type = other RNA
tttcgactggatacaccttgatgttaaacgtcctacctccg

SEQ ID NO: 12 - RNA, 49 nt, synthetic construct
  source 1..49: mol_type = other RNA
agacagtttcgactggatacaccttgatgttaaacgtcctaccccgcca

SEQ ID NO: 13 - RNA, 50 nt, synthetic construct; identical to SEQ ID NO: 1
  source 1..50: mol_type = other RNA

SEQ ID NO: 14 - RNA, 80 nt, synthetic construct
  source 1..80: mol_type = other RNA
acgtaaactgcggacagttactcgattcaaagacagtttcgactggatacaccttgatgttaaacgtcctacctccgcca

SEQ ID NO: 15 - RNA, 21 nt, synthetic construct
  source 1..21: mol_type = other RNA
taaactgcggacagttactcg

SEQ ID NO: 16 - DNA, 15 bp, synthetic construct
  source 1..15: mol_type = other DNA
tcaaggtgtatccag